(19) United States
(12) Patent Application Publication
(10) Pub. No.: US 2016/0296762 A1
(43) Pub. Date: Oct. 13, 2016

(54) APPARATUSES AND METHODS FOR CLASSIFICATION OF ELECTROCARDIOGRAM SIGNALS DURING CARDIOPULMONARY RESUSCITATION

(71) Applicant: UNIVERSITY OF WASHINGTON THROUGH ITS CENTER FOR COMMERCIALIZATION, Seattle, WA (US)

(72) Inventors: Alampallam R. Ramachandran, Seattle, WA (US); Lawrence Sherman, Seattle, WA (US); Jason Coult, Seattle, WA (US); Peter Kudenchuk, Seattle, WA (US); Allison Chin, Seattle, WA (US); Christopher Neils, Seattle, WA (US)

(73) Assignee: UNIVERSITY OF WASHINGTON THROUGH ITS CENTER FOR COMMERCIALIZATION, Seattle, WA (US)

(21) Appl. No.: 15/026,952

(22) PCT Filed: Oct. 3, 2014
(86) PCT No.: PCT/US14/59108

§ 371 Date: Apr. 1, 2016

Related U.S. Application Data
(60) Provisional application No. 61/886,198, filed on Oct. 3, 2013.

(51) Int. Cl. A61N 1/39; A61N 1/04; A61B 5/046; A61B 5/0402; A61B 5/00

(52) U.S. Cl. CPC A61N 1/3987; A61B 5/0402; A61B 5/4836; A61B 5/046; A61N 1/046; A61N 1/3925

(57) ABSTRACT

Examples of systems, apparatuses, and methods for classification of electrocardiogram signals during cardiopulmonary resuscitation are described. An example system may include a defibrillator comprising an electrocardiogram analyzer. The electrocardiogram analyzer may be configured to apply a prediction modeling technique to an electrocardiogram signal to generate a predicted signal. The electrocardiogram signal may be captured from a patient undergoing cardiopulmonary resuscitation. The electrocardiogram analyzer may be further configured to subtract the predicted signal from the electrocardiogram signal to generate an error signal and to classify a rhythm of the electrocardiogram signal as one of a shockable rhythm or non-shockable based on the error signal. Decision parameters derived from the signals may be used in conjunction with a machine learning technique to classify the electrocardiogram signal.

### CROSS-REFERENCE TO RELATED APPLICATION(S)

This application claims the benefit of the earlier filing dates of U.S. Provisional Application No. 61/886,198, filed Oct. 3, 2013, entitled “Classification of Electrocardiogram Signals Using Linear Predictive Coding Error,” which is hereby incorporated by reference in its entirety for any purpose.

### TECHNICAL FIELD

Examples described herein relate generally to classification of electrocardiogram signals during cardiopulmonary resuscitation.

### BACKGROUND

Cardiopulmonary resuscitation (CPR), combined with defibrillation, is an essential treatment of cardiac arrest and involves chest compressions designed to perfuse the heart, brain and other organs during the arrest. In some instances, automatic external defibrillators (AED) may be designed to analyze an electrocardiogram (ECG) signal during a cardiac arrest through two electrode pads attached to the chest of the patient in order to determine whether to provide a shock to the patient via the two pads. An ECG signal provides an indication of electrical activity of the heart. The two pads attached to the patient may detect electrical pulses generated by the polarization and depolarization of cardiac tissue, and translates the electrical pulses into a waveform. The waveform can be used to measure rate and regularity of heartbeats, as well as size and position of the chambers, the presence of any damage to the heart, and the effects of drugs or devices used to regulate the heart.

During a cardiac arrest, an AED may analyze the ECG signal to detect whether the patient's heart is exhibiting a shockable ECG rhythm. An example of a shockable rhythm may include ventricular fibrillation (i.e., a condition where there is uncoordinated contraction of the cardiac muscle of the ventricles of the heart, causing the cardiac muscles to quiver rather than contract in a coordinated fashion). Examples of non-shockable ECG rhythms may include asystole (i.e., flatline or state of no cardiac electrical activity), organized cardiac electrical activity (including rhythms that produce blood flow), or pulseless electrical activity (i.e., electrical signals indicate heart rhythm, but no pulse is produced). Thus, prior to delivering a shock, an AED must first determine if the underlying ECG signal indicates a shockable rhythm with reasonable certainty, to avoid administering a shock to a patient with a non-shockable rhythm.

Conventional AEDs instruct a responder to provide CPR chest compressions and artificial ventilation during the arrest. Provision of CPR introduces artifacts into an ECG signal, obscuring the ability of the AED to detect an ECG rhythm of the heart of the patient. Thus, conventional AEDs periodically require the responder to cease CPR (e.g., for 7 or more seconds) to allow for analysis of the ECG rhythm via the ECG signal. If a shockable rhythm is detected, the AED may deliver a shock. Cessation of CPR for analysis, even for a short while, may significantly reduce chances of survival due to, among other issues, loss of perfusion pressure.

### SUMMARY

Examples of systems, apparatuses, and methods for classification of electrocardiogram signals during cardiopulmonary resuscitation are described herein. An example system may include a defibrillator comprising an electrocardiogram analyzer. The electrocardiogram analyzer may be configured to apply a prediction modeling technique to an electrocardiogram signal to generate a predicted signal. The electrocardiogram signal may be captured from a patient undergoing cardiopulmonary resuscitation. The electrocardiogram analyzer may be further configured to subtract the predicted signal from the electrocardiogram signal to generate an error signal and to classify a rhythm of the electrocardiogram signal as one of a shockable rhythm or a non-shockable rhythm based on the error signal.

An example method may include generating a residual error signal by subtracting a predicted signal from an electrocardiogram signal. The electrocardiogram signal may include artifacts associated with a patient undergoing cardiopulmonary resuscitation. The example method may further include generating decision parameters based on the residual error signal. The decision parameters may indicate characteristics of the residual error signal. The example method may further include determining a respective probability value associated with each of at least one electrocardiogram rhythms based on the decision parameters using a decision module, and classifying the electrocardiogram signal based on the probability value. The decision module may be trained using previously captured electrocardiogram signals.

Another example method may include applying a predictive modeling technique to an electrocardiogram signal to generate a predicted signal. The electrocardiogram signal may include artifacts associated with a patient undergoing cardiopulmonary resuscitation. The example method may further include subtracting the predicted signal from the electrocardiogram signal to generate an error signal. The example method may further include classifying a rhythm of the electrocardiogram signal as one of a shockable rhythm or non-shockable rhythm based on the error signal.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several examples in accordance with the disclosure and are, therefore, not to be considered limiting of its scope, the disclosure will be described with additional specificity and detail through use of the accompanying drawings, in which:

FIG. 1 is an exemplary illustration of an automatic external defibrillator system applied to a patient according to an embodiment of the present disclosure.

FIG. 2 is a block diagram of a defibrillation system according to an embodiment of the present disclosure.

FIG. 3 is a block diagram of an electrocardiogram analyzer according to an embodiment of the present disclosure.

FIG. 4 is a flow chart of an exemplary method for classifying an electrocardiogram rhythm according to an embodiment of the present disclosure.

FIG. 5 is a flow chart of an exemplary method for classifying an electrocardiogram rhythm according to an embodiment of the present disclosure.

FIG. 6 is a block diagram illustrating an example computing device that includes an ECG analyzer an embodiment of the present disclosure.

### DETAILED DESCRIPTION

In the following detailed description, reference is made to the accompanying drawings, which form a part hereof. In the drawings, similar symbols typically identify similar components, unless context dictates otherwise. The illustrative examples described in the detailed description, drawings, and claims are not meant to be limiting. Other examples may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are implicitly contemplated herein.

Examples described herein relate generally to apparatuses, systems, and methods for classification of rhythms of ECG signals during a medical procedure, such as during cardiopulmonary resuscitation or artificial ventilation. While the examples described herein are primarily discussed in the context of automatic external defibrillators, it will be understood that the apparatuses, systems, and methods disclosed are equally applicable and can be used in the context of any other therapeutic or clinical device, such as with hospital monitors, implantable defibrillators, or other defibrillators with a capability of classifying a rhythm of an ECG signal. Generally, examples of the present invention may be used with any ECG signal. Accordingly, the particular examples provided herein are for illustration purposes only and are not to be taken in a limiting sense.

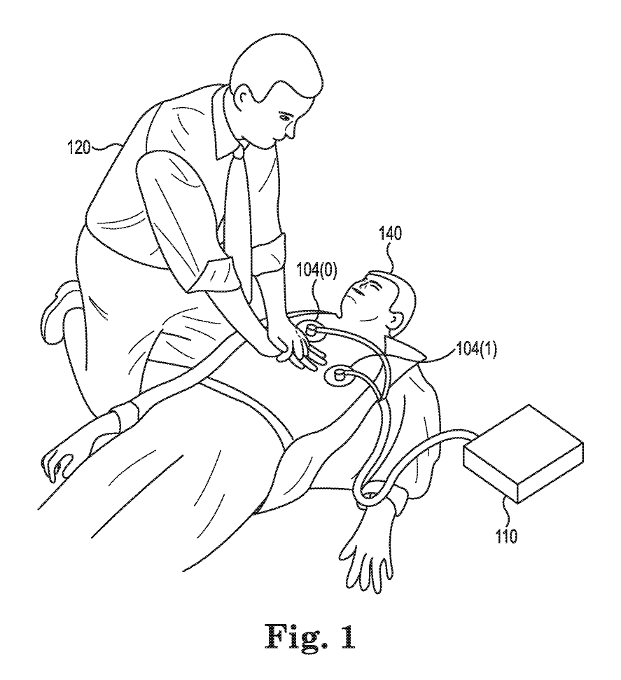

FIG. 1 is an illustration of a responder 120 performing CPR on a patient 140 that is connected to an AED 110. In this scenario, the patient 140 may be exhibiting signs of cardiac arrest. The responder 120 may be a person that is trained in proper CPR techniques. In this example, the patient 140 may have two electrodes 104(0-1) applied to his/her chest. The two electrodes 104(0-1) may be attached to the skin of the patient 140 at conventional locations, such as one electrode 104(0) applied under the right collar bone and the other electrode 104(1) applied to left lower chest. The two electrodes 104(0-1) may be coupled to the AED 110 via a cable.

The AED 110 may detect an ECG signal from the patient 140 via the two electrodes 104(0-1), including while the responder 120 is performing CPR. The AED 110 may analyze the ECG signal to classify the ECG rhythm of the patient 140 as shockable or non-shockable. The AED 110 may apply high-voltage (e.g. 1,300-1,800 volts) shocks responsive to the classification of a shockable rhythm. While the AED is connected to the two electrodes 104(0-1) to detect the ECG signal, the responder 120 may perform CPR by applying downward forces or compressions to the sternum of the patient 140. In some instances, CPR may also include the responder 120 blowing air into the mouth or nose of the patient 140 by mouth-to-mouth or mouth-to-nose breathing. In some examples, the AED 110 may prompt the responder 120 to stop CPR to allow for a shock to be administered to the patient 140.

The AED 110 may include an ECG analyzer to classify the ECG rhythm of the patient 140 as shockable or non-shockable while the responder 120 is administering chest compressions. Normally, chest compressions may introduce artifacts into the ECG signal, which may mask or obscure the underlying ECG rhythm, making classification of an ECG rhythm of the patient 120 difficult. The ECG analyzer of the AED 110 may apply signal processing techniques to the ECG signal to classify an ECG rhythm despite the artifacts introduced by the chest compressions. In some examples, the ECG analyzer may use a predictive modeling technique to generate a predicted signal from a captured portion of an ECG signal (e.g., a clip), and to subtract the predicted signal from the ECG signal to provide an error signal E. An example of a predictive modeling technique may include linear predictive coding (LPC) filtering techniques. In some examples, the ECG signal clip may be less than 5 seconds, such as 3.8 seconds. The ECG analyzer may further process the ECG signal, and the error signal E to derive parameters for use in the ECG classification method. In some embodiments, the AED 110 may apply a bandpass filter to the error signal E to generate a bandpass error signal EBP.

The ECG analyzer may generate parameters from the ECG signal S, the error signal E, and/or the bandpass error signal E which can be used to isolate or enhance/highlight certain characteristics of the signals. The ECG analyzer may classify the ECG signal clip as a shockable or non-shockable rhythm based on the derived parameters. In some embodiments, the decision module may include an artificial neural network. The artificial neural network may be trained using previously captured and classified ECG signal clips from multiple patient recordings. In some examples, the neural network may be trained using more than 1000 sample ECG signal captures. In some examples, the ECG analyzer may also be capable of classifying an ECG signal clip that does not contain artifacts related to CPR or another medical procedure.

In some embodiments, the ECG analyzer may include other or different decision making methodologies. While the above describes the classification of an ECG rhythm in an AED 110, the classification may be performed in other devices, such as an implantable defibrillator or an ECG monitor in a hospital setting that constantly or periodically monitors ECG signals to classify ECG rhythms for evaluations over time, and/or monitors ECG rhythms during a medical event.

While AED 110 is described as an automatic external defibrillator, which is generally designed for small physical size, light weight, and relatively simple user interface capable of being operated by personnel without high training levels, in other embodiments, the AED 110 may additionally or alternatively include other defibrillators, such as a manual defibrillator, an implantable defibrillator, a paramedic defibrillator, and/or a clinical defibrillator. Generally, paramedic or clinical defibrillators may be carried by an emergency medical service (EMS) responder, and tend to be larger, heavier, and have a more complex user interface capable of supporting a larger number of manual monitoring and analysis functions.

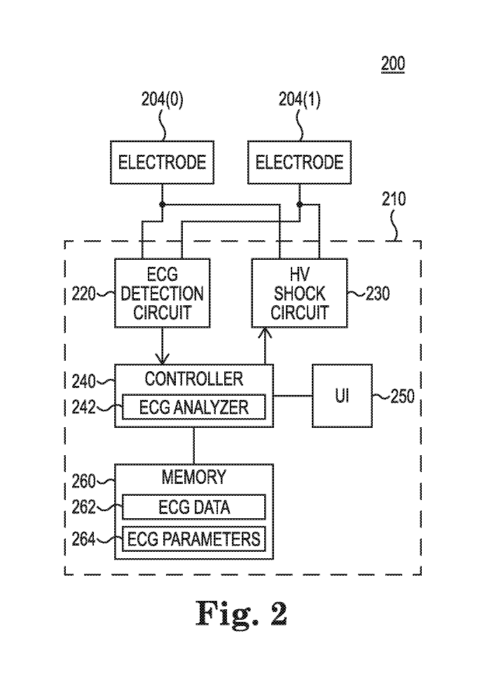

FIG. 2 is a block diagram of defibrillation system 200 according to an embodiment of the disclosure. The defibrillation system 200 may include a pair of electrodes 204(0-1) coupled to an AED 210. The AED 210 may be implemented in the AED 110 of FIG. 1.

The AED 210 may be include an ECG detection circuit 220 coupled to the pair of electrodes 204(0-1). The pair of electrodes 204(0-1) may be connected across the chest of a patient, such as the patient 140 of FIG. 1. The ECG detection circuit 220 may amplify, buffer, and/or filter and digitize an electrical ECG signal generated by the patient's heart to produce a stream of digitized ECG samples. The ECG detection circuit 220 may provide the digitized ECG samples to a controller 240. The controller 240 may include an ECG analyzer 242 that performs an analysis of a subset of digitized EGC samples (e.g., an ECG signal clip) to classify the ECG rhythm of the patient as shockable or non-shockable. If a shockable rhythm is detected (e.g., in combination with determination of a treatment regimen that indicates immediate defibrillation shock), the controller 240 may send a signal to high voltage (HV) shock circuit 230 to charge in preparation for delivering a shock. The AED 210 may include a user interface 250 that provides an indication to the controller 240 to administer the shock responsive to a user input. For example, responsive to receiving an indication that a user has pressed a shock button on the user interface 250, the controller 240 may command the HV shock circuit 230 to initiate a shock of the patient via the pair of electrodes 204(0-1). The AED 210 may further include a memory 260 that is configured to store ECG signal data used by and ECG parameters generated by the ECG analyzer 242.

In operation, the pair of electrodes 204(0-1) may be attached to a patient experiencing a medical event, such as cardiac arrest. The ECG detection circuit 220 may receive an ECG signal indicating electrical activity of the heart of the patient via the pair of electrodes 204(0-1). The ECG detection circuit 220 may continuously sense the ECG signal of the patient, including while the patient is receiving CPR or other medical care. The ECG detection circuit 220 may apply signal processing techniques the ECG signal to provide digitized samples of the ECG signal to the controller 240. In some examples, the ECG signal may be sampled at 250 Hz, but other sample rates may be used. The ECG analyzer 242 may analyze a predetermined number of samples (e.g., a clip) of the digitized ECG signal (e.g., over a specified time length) to classify the ECG rhythm of the patient as shockable or non-shockable. An example of a shockable rhythm may include ventricular fibrillation. Examples of non-shockable rhythms may include asystole (e.g., flatline or state of no cardiac electrical activity), organized cardiac activity (e.g., normal sinus rhythm), or pulseless electrical activity (e.g., electrical signals indicate heart rhythm, but no pulse is produced). If a shockable classification is determined, the controller 240 may send a command to the HV shock circuit 230 to begin charging. Responsive to an input at the user interface 250, the HV shock circuit 230 may release the high voltage to the electrodes 204(0-1) to administer a shock to a patient.

While a patient is being administered CPR, classification of the ECG rhythm of the ECG signal clip by the ECG analyzer 242 may include preprocessing of the digitized samples of the ECG signal to provide a preprocessed ECG signal S. For example, the ECG analyzer 242 may apply a window function (e.g., tapered cosine window) and/or a bandpass filter to the digitized samples of the ECG signal. In some examples, the upper corner frequency may be less than 50 or 60 Hz to filter out electrical noise, and the lower corner frequency may be less than 1 Hz to remove baseline drift in the signal. The preprocessing may filter out noise and other extraneous data from the digitized samples of the ECG signal.

The ECG analyzer 242 may apply a predictive modeling technique to generate a predicted signal from an ECG signal clip. An example of the predicted modeling technique may include an LPC filter. In an embodiment that uses LPC filter techniques, the LPC filter may be adapted for ECG processing to the preprocessed ECG signal S to generate the predicted signal. LPC filtering is typically used in audio signal and speech processing to represent the spectral envelope of a digital signal of speech in compressed form using information of a linear predictive model. Thus, LPC filter techniques applied to the preprocessed ECG signal may be adapted for ECG signal characteristics, which may encompass a different spectral envelope than human speech, to generate LPC coefficients. In some examples, the ECG analyzer 242 may apply obtain 2^{nd} order LPC filter coefficients, but other orders may be used.

Use of the LPC filter is different from some other processing approaches in that the LPC filter may adapt differently to each ECG signal clip. Typically, a filter design may be fixed and perform the same operations on all input signals. The LPC filter may adapt to create a different filter for each ECG signal clip, and thus, the difference between the ECG signal S and the predicted signal may be more closely representative of random activity a specific ECG signal clip. In some examples, the LPC filter may be applied to an entire ECG signal clip. In other embodiments, the LPC filter may be applied to overlapping windowed sections of the ECG signal slip.

The ECG analyzer 242 may subtract the predicted signal from the preprocessed ECG signal S to generate an error signal E. The ECG analyzer 242 may further apply a bandpass filter to the error signal E to generate the bandpass error signal EBP.

The ECG analyzer 242 may generate decision parameters based on the ECG signal S, the error signal E, and/or the bandpass error signal EBP. The decision parameters may be stored at the memory 260, and/or may be stored at the ECG analyzer 242. One of skill in the art would appreciate that the decision parameters may be generated using different methods or inputs (e.g., filter orders, constraints, cutoffs, etc.), and/or that the decision parameters may be derived in different ways. Examples of the decision parameters may include standard deviations, standard deviation ratios of the signals and/or frequency subbands of the signals, indications of magnitude within the signals or frequency subbands of the signals, indications of frequency of common or similar values within the signals or frequency subbands of the signals, etc.

The decision parameters may be provided to a decision module of the ECG analyzer 242 to classify the ECG rhythm of the patient as shockable or non-shockable. The decision module may be an artificial neural network trained using previously captured and classified ECG signals. Other decision module implementations based on machine learning may be used, such as support vector machines or logistic regression. In some examples, the decision module may generate probabilities for various ECG signal rhythms, and may select the ECG rhythm having the highest probability. For example, the decision module may generate probabilities for ventricular fibrillation, asystole, and/or organized electrical activity. The decision module may provide a shockable or non-shockable determination based on whether the selected ECG rhythm is a shockable rhythm or a non-shockable rhythm. In some examples, the ECG analyzer 242 may also be capable of classifying an ECG signal clip that does not contain artifacts related to CPR or another medical procedure.

The controller 240 may initiate a shock of the patient based on the classification of the ECG rhythm by the ECG analyzer 242 (e.g., initiate a shock responsive to a shockable rhythm being detected). The shock may be delivered via the pair of electrodes 204(0-1) using an electrical charge stored at the HV shock circuit 230. An ability to classify the ECG rhythm of a patient while CPR or another medical procedure such as artificial ventilation is being performed may increase a likelihood of a patient experiencing a medical event to recover from the medical event by maintaining perfusion pressure, especially to brain tissue and cardiac muscle tissue, in the patient while the ECG rhythm is being analyzed.

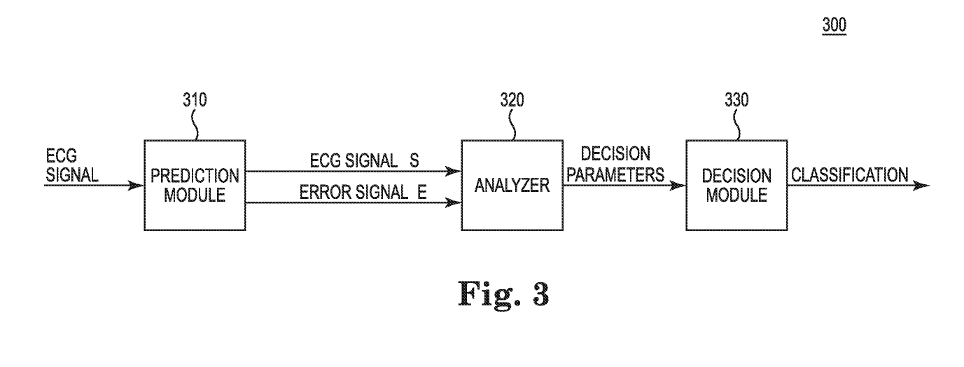

Other techniques or algorithms for generating the error signal may be implemented in addition to or in lieu of the LPC filter technique. For example, in another embodiment, the error signal, E, may be obtained by using principal component analysis (PCA), performing eigenvalue decomposition of the signal, S, to first obtain an intermediate signal composed of only those eigenvectors that correspond to the larger eigenvalues, and then subtracting this intermediate signal from S to obtain the residual or error signal, E. The intermediate signal is similar to the predicted signal in the case of the LPC approach. FIG. 3 is a block diagram of an ECG analyzer 300 according to an embodiment of the disclosure. The ECG analyzer 300 may be implemented in the ECG analyzer 242 of FIG. 2. The ECG analyzer 300 may include a prediction module 310, an analyzer 320, and a decision module 330. The prediction module 310 may receive a digitized ECG signal clip and preprocess the digitized ECG signal clip to provide a preprocessed ECG signal S. The prediction module 310 may apply a predictive modeling technique to generate a predicted signal from an ECG signal clip. The prediction module 310 may subtract the predicted signal from the preprocessed ECG signal S to generate a residual error signal E. Note that one of skill in the art would understand that the preprocessed ECG signal S may be subtracted from the predicted signal is within the scope of this disclosure. The preprocessed ECG signal S and the error signal E may be provided to the analyzer 320. The analyzer 320 may apply a bandpass filter to the error signal E to generate a bandpass error signal EBP, and may generate decision parameters based on the preprocessed ECG signal S, the error signal E, and the bandpass error signal EPB. The decision parameters may indicate characteristics of the ECG signal that are used by the decision module 330 to classify the ECG rhythm of the ECG signal clip. The decision module 330 may include an artificial neural network configured to receive the decision parameters, and to generate a classification of the ECG signal based on application of the decision parameters within the artificial neural network.

In operation, the ECG analyzer 300 may be configured to classify an ECG signal clip that is received from a patient undergoing CPR (e.g., or another procedure that introduces artifacts into the ECG signal, such as breathing or artificial ventilation). Thus, while a patient is being administered CPR, the prediction module 310 may preprocess the digitized ECG signal clip to generate a preprocessed ECG signal S. For example, the prediction module 310 may apply a window function (e.g., tapered cosine window) and/or a bandpass filter to the digitized samples of the ECG signal. In some embodiments, an upper corner frequency may be less than 50 or 60 Hz to remove environmental electrical noise (e.g., from power system). The preprocessing may filter out noise and other extraneous data from the digitized samples of the ECG signal.

The prediction module 310 may further apply a predictive modeling technique to the preprocessed ECG signal S to generate a predicted signal. The prediction module 310 may subtract the predicted signal from the preprocessed ECG signal S to generate a residual error signal E. An example of a predictive modeling technique may include an LPC filter and/or another predictive modeling technique. In embodiments using LPC filter techniques, the LPC filter may be adapted for ECG processing. In some examples, the prediction module 310 may apply 2^{nd} order LPC filter coefficients, but other orders may be used. Alternatively, PCA may use orthogonal transformation to convert an ECG signal clip into a set of values of linearly uncorrelated variables, e.g., principal components, and produce an modeled intermediate signal that can be subtracted from the preprocessed signal S to generate the error signal E.

The predictive modeling technique used to generate the predicted signal may provide the predicted signal with most of the CPR artifacts, as well as any other low frequency behavior in the preprocessed ECG signal S, removed in an adaptive way that changes or adapts based on characteristics of the ECG signal S. This may provide the error signal E that is strongly affected by the presence of characteristics of an organized ECG rhythm (e.g., QRS complexes), as well as smoothness to allow for detection of asystole and ventricular fibrillation (e.g., asystole is smoother than ventricular fibrillation). That is, quantifying the error signal may help distinguish these ECG rhythms from each other within an ECG signal clip.

The analyzer 320 may apply a bandpass filter to the error signal E to provide a bandpass error signal EBP. In some embodiments, the bandpass may filter the error signal E to isolate a frequency band of the error signal E having a bandwidth between 10 Hz to 50 Hz. The lower frequency of the filter may be between 5 Hz and 15 Hz. In other embodiments, the prediction module 310 may generate several error signal E bands centered at different frequencies using multiple bandpass filters.

The analyzer 320 may further generate decision parameters based on the digitized ECG signal, the preprocessed ECG signal S, the error signal E, and/or the bandpass error signal EBP. The decision parameters may indicate characteristics of the signals that are used by the decision module 330 to classify the ECG rhythm of the patient as shockable or non-shockable. The ECG rhythms, while generally exhibiting distinctly different characteristics in an ideal case, operate on a continuum in the real world that may include overlap of various characteristics or indicators. Thus, it may be challenging to classify an ECG rhythm using a single indicator or characteristic. For example, VF or organized electrical activity may result in a higher energy error signal E than asystole due to difficulty of the predictive modeling technique's ability to model either of these rhythms in a signal, including a signal that is largely artifacted by CPR (or another medical procedure). However, because the error of the energy signal for VF and organized electrical activity may largely overlap, it may prove unreliable to use the energy of the error signal E to distinguish between VF and organized electrical activity. The decision parameters may attempt to isolate or enhance/highlight various characteristics that collectively may prove to be valuable indicators to identify a highest probability ECG rhythm. Examples of the decision parameters are described below. One of skill in the art would appreciate that the decision parameters may be generated using different methods or inputs (e.g., filter orders, constraints, cutoffs, etc.), and/or that the decision parameters may include all or any sub-combination of the described parameters, and/or additional and/or different parameters than the described parameters.

For example, the analyzer 320 may determine standard deviations of the preprocessed ECG signal S (e.g., std_{S}), the error signal E (e.g., std_{E}), and/or the bandpass error signal EPB (e.g., std_{EPB}). The ECG analyzer 242 may further determine a ratio of the standard deviation of the error signal E to the standard deviation of the preprocessed ECG signal S (e.g., std_{E}/std_{S}). The standard deviations may estimate how much energy is in a signal, and the std_{E}/std_{S} ratio may indicate a portion of the energy of that could not be modeled by the predictive model.

Additional parameters may be determined to further distinguish between various ECG rhythms. For example, one or more of the following parameters may be generated from the error signal E and/or the bandpass error signal EBP. Note that the use of E in Table 1 may refer to either or both of the error signal E and the bandpass error signal EBP.

TABLE 1
Example Decision Parameters

${sumInvAbs}_{ E} = \sum \frac{1}{\vert E \vert} , in some examples a median filter may be applied$
to E prior to calculating sumInvAbs_{E}. Further, in some examples, iso-
lated zero values may be removed. In some examples, log(sumInvAbs_{E})
may be used instead of sumInvAbs_{E}.
sumInvNormAbs_{E} = sumInvAbs_{E} * max(|E|)
In some examples, the standard deviation or variance of E may be used in
place of max(|E|) to normalize sumInvAbs_{E}.
In some examples, log(sumInvNormAbs_{E}) may be used instead of
sumInvAbs_{E}.
SmoothInvAbs_{E} may be calculated as follows:

| | |
|---|---|
| 1. | ${filt}_{ E} = filter ( \frac{1}{\vert E \vert} ) ; filter may be a smoothing filter such as a boxcar$ |
| | or hamming filter. |
| 2. | filt_{E} is sorted by absolute magnitude and subsortfilt_{E} = largest x% of |
| | values, where x% may be 50% or another percentage. |
| 3. | ${smoothInvAbs}_{ E} = log ( \sum subsortfiltE )$ |
| | ${smoothInvNormAbs }_{E} = log ( \sum ( {subsortfilts}_{ E} * max ( \vert E \vert ) ) )$ |

probAbsMax_{E} may be calculated as follows:

| | |
|---|---|
| 1. | logabs_{E} = log(\|E\|). |
| 2. | Generate a histogram using logabs_{E}. |
| 3. | probAbsMax_{E} = maximum point in histogram. |

Note: In some embodiments, to avoid random noise, the histogram may
be smoothed prior to selecting the value for probAbsMax_{E}. For example,
the histogram may be represented as probability distribution, and the
estimate of the maximum point from the distribution curve may be drawn
for probAbsMax_{E}. In one embodiment, a −log(|E|) (e.g., or another
means) may be taken and fit to a gamma distribution. Then, the value of
the −log(|E|) values that corresponds to the maximum value of the
probability distribution is selected as probAbsMax_{E}. In some examples,
zeroes may be removed and negative values may be eliminated by
applying a constant prior to determining the −log(|E|) values. The
gamma distribution's parameter values, alpha and beta, may also be used
as parameters, because they also quantify the distribution shape. Other
probability distributions, such as the Rayleigh distribution as an example,
may also be used to characterize the histogram shape of the |E| values.
probNormAbsMax_{E} may be calculated as follows:

| | |
|---|---|
| 1. | ${logNormabs}_{ E} = log ( \frac{\vert E \vert}{max ( \vert E \vert )} ) .$ |
| 2. | Generate a histogram using logNormabs_{E}. |
| 3. | probAbsMax_{E} = maximum point in histogram. |

Note: In some embodiments, to avoid random noise, the histogram may
be smoothed prior to selecting the value for probNormAbsMax_{E}. For
example, the histogram may be represented as probability distribution,
and the estimate of the maximum point from the distribution curve may be
$drawn for {probNormAbsMax}_{ E} . In one embodiment , a - log ( \frac{\vert E \vert}{max ( \vert E \vert )} )$
(e.g., or anther means) may be taken and fit to a gamma distribution. Then,
$the value of the - log ( \frac{\vert E \vert}{max ( \vert E \vert )} ) v alues that corresponds to the maxi \text{-}$
mum value of the probability distribution is selected as probAbsMax_{E}. In
some examples, zeroes may be removed and negative values may be elimi-
$nated by applying a constant prior to determining the - log ( \frac{\vert E \vert}{max ( \vert E \vert )} )$
values. The gamma distribution's parameter values, alpha and beta, may
also be used as parameters, because they also quantify the distribution
shape. Other probability distributions, such as the Rayleigh distribution as
an example, may also be used to characterize the histogram shape of the
|E| values.
dcHilbert_{EBP} may be calculated as follows:

| | |
|---|---|
| 1. | Generate the Hilbert envelope of EBP. |
| 2. | dcHilbert_{EBP} = the first coefficient (e.g., DC component) of the |
| | Fourier transform of the Hilbert envelope. |

As previously described, the parameters may isolate characteristics of the error signal E and/or the bandpass error signal EBP that may be useful in classifying the highest probability ECG rhythm of the ECG signal clip. As such, because the ECG rhythms operate on continuums, the following discussion may reflect a general case, and may not be true for all cases.

sumInvAbs_{E} may be indicative of low levels of a signal. Asystole, which is a generally flatline ECG signal, may have more low levels than VF or organized electrical activity, and thus, may result in a larger parameter value, and VF, which is uncoordinated electrical activity (e.g., essentially uncoordinated noise) may have a lowest value as the predictive model may have difficulty modeling uncoordinated noise on a signal. sumInvNormAbs_{E} may be similar to sumInvAbs_{E}, but the normalization may result in the asystole error having a small value, with organized electrical activity resulting in a higher parameter value, and VF resulting in a lowest parameter value. This may be due to asystole being normalized by a small maximum absolute value, while organized electrical activity is normalized by a relatively large maximum absolute value, and VF normalized by an intermediate maximum absolute value. A similar analysis may apply for the smoothInvAbs_{E} and smoothInvNormAbs_{E} parameters.

probAbsMax_{E} may estimate a range of magnitudes of the most frequent error signal E values, which may be related to the ECG rhythm due to the ability or inability of the predictive model to predict the behavior of certain ECG rhythms. Because maximum values of a histogram may generally correlate to a relatively low signal values (e.g., since an ECG signal may mostly reside at a low signal value regardless of an ECG rhythm), the histogram may be manipulated in such a way to include additional values other than the absolute lowest values to distinguish between the ECG rhythms. probAbsNormMax_{E} may provide another data point to further distinguish between the ECG rhythms based on frequent error signal E values.

dcHilbert_{EBP} may indicate energy around a center frequency of the bandpass filter used to generate the bandpass error signal, which may be generally larger for VF than for asystole or organized electrical activity.

The decision parameters may be provided to the decision module 330 to classify the ECG rhythm of the patient as shockable or non-shockable. The decision module may be an artificial neural network trained using previously captured and classified ECG signals. The artificial neural network may compute the prediction levels of three signal classes (e.g., VF (shockable), organized electrical activity (non-shockable), and asystole (non-shockable)) based on the decision parameters computed from the preprocessed ECG signal S, the error signal E, and the bandpass error signal EBP. The artificial neural network may include coefficients/weights determined based on the training via the pre-classified, CPR-artifacted ECG signals. Training may also occur using non-artifacted ECG signals (e.g., not artifacted by CPR, artificial ventilation, and/or anther medical procedure). The decision module 330 may select the ECG rhythm classification having a highest probability or a probability in relation to a predefined decision threshold. Based on the selected ECG rhythm, the decision module 330 may provide a shockable or non-shockable indication at an output.

In some embodiments, rather than providing the three-class probability output, the artificial neural network may provide a shockable rhythm/non-shockable rhythm decision by ignoring the asystole and organized electrical activity predictions (e.g., only observing the probability of VF). The artificial network may also be trained on shockable versus non-shockable signals (as opposed to asystole vs organized electrical activity vs VF), to produce a shockable/non-shockable decision based on the probability value associated with VF. The number of hidden layers in the network, the number of hidden nodes in each hidden layer, the number of input parameters, and the number of rhythm classes may be varied to optimize performance depending on the specific application of the artificial neural network.

In other embodiments, the decision module 330 implementations based on machine learning other than or in addition to the artificial neural network may be used, such as support vector machines, deep learning neural networks, or logistic regression. Further, in some examples, the ECG analyzer 300 may also be capable of classifying an ECG signal clip that does not contain artifacts related to CPR or another medical procedure.

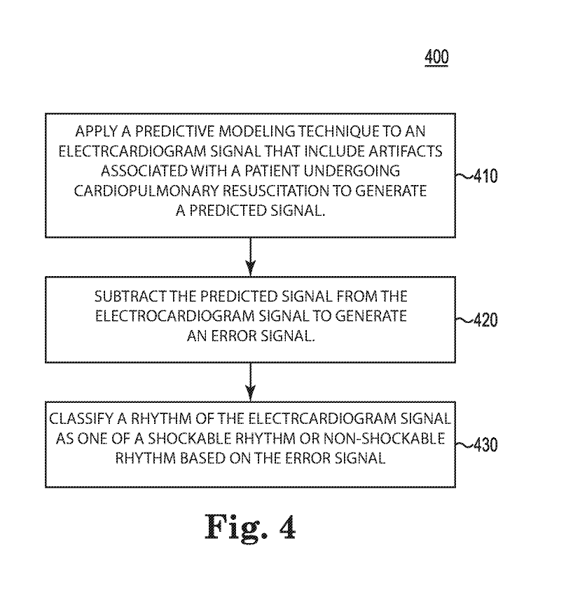

FIG. 4 is a flow chart of an exemplary method 400 according to the present disclosure. The method 400 may be implemented in the AED 100 of FIG. 1, the controller 240, the ECG analyzer 242, and/or the memory 260 of FIG. 2, the ECG analyzer 300 of FIG. 3, or any combination thereof.

The method 400 may include applying a predictive modeling technique to an ECG signal to generate a predicted signal, at 410. The ECG signal may include artifacts associated with a patient undergoing CPR. Application of the predictive modeling technique may be performed by the ECG analyzer 242 of FIG. 2 and/or the prediction module 310 of FIG. 3. The predictive modeling technique may include an LPC filter, or another predictive modeling technique. In some embodiments, the method 400 may further include preprocessing of the ECG signal prior to applying predictive modeling technique to the ECG signal. For example, the method 400 may include applying a window function (e.g., tapered cosine window) and/or a bandpass filter to the ECG signal. The preprocessing may filter out noise and other extraneous data from the digitized samples of the ECG signal.

The method 400 may further include subtracting the predicted signal from the ECG signal to provide an error signal, at 420. Subtraction of the predicted signal from the ECG signal may be performed by the ECG analyzer 242 of FIG. 2 and/or the prediction module 310 of FIG. 3. In some embodiments, the method may further include applying a bandpass filter to the error signal to generate a bandpass error signal. Application of the bandpass filter to the error signal may be performed by the ECG analyzer 242 of FIG. 2 and/or the analyzer 320 of FIG. 3.

The method 400 may further include classifying the electrocardiogram signal as one of a shockable rhythm or a non-shockable rhythm, at 430. Classification of the ECG signal may be performed by the ECG analyzer 242 of FIG. 2 and/or the analyzer 320 and the decision module 330 of FIG. 3. In some embodiments, the method 400 may further include generating decision parameters from the ECG signal, the error signal, and/or the bandpass error signal. The decision parameters may indicate characteristics of the signals that are used to classify the ECG rhythm of the patient as shockable or non-shockable. Examples of the decision parameters may include std_{S}, std_{E}, std_{EPB}, a std_{E}/std_{S} ratio, sumInvAbs_{E}, sumInvAbs_{E}, smoothInvAbs_{E}, smoothInvNormAbs_{E}, probAbsMax_{E}, probAbsNormMax_{E}, dcHilbert_{EBP}, different additional parameters, or any combination thereof.

In some embodiments, the method 400 may further include generating probabilities associated with each of various ECG rhythms (e.g., VF, asystole, or organized electrical activity) based on the decision parameters. The method 400 may further include selecting an ECG rhythm having a highest probability.

In some embodiments, the method 400 may further include generating a respective probability associated with each of a shockable rhythm and a non-shockable rhythm based on the decision parameters. The method 400 may further include selecting one of a shockable rhythm or non-shockable rhythm based on that which has a higher probability.

Generating the probabilities may be performed by the ECG analyzer 242 of FIG. 2 and/or the decision module 330 of FIG. 3. Generation of the ECG rhythm probabilities and/or the shockable/non-shockable rhythm probabilities may be determined using machine learning techniques such as an artificial neural network, support vector machines, logistic regression, or any combination thereof, which may be trained using pre-classified ECG signal clips.

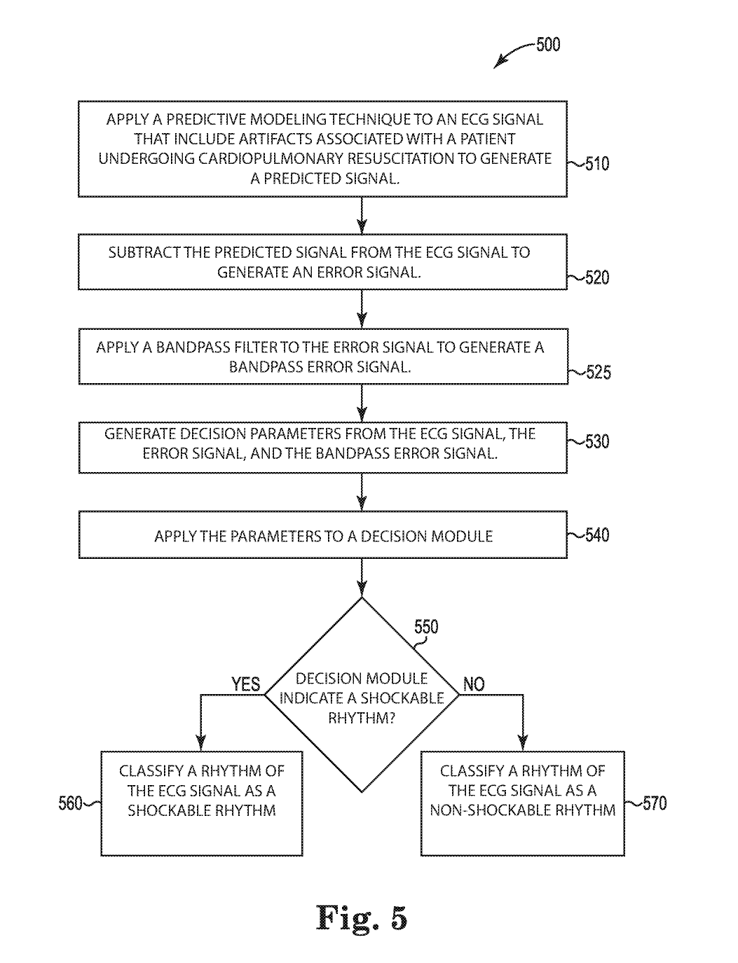

FIG. 5 is a flow chart of an exemplary method 500 according to the present disclosure. The method 500 may be implemented in the AED 100 of FIG. 1, the controller 240, the ECG analyzer 242, and/or the memory 260 of FIG. 2, the 300 of FIG. 3, or any combination thereof.

The method 500 may include applying a predictive modeling technique to an ECG signal to generate a predicted signal, at 510. The ECG signal may include artifacts associated with a patient undergoing CPR. Application of the predictive modeling technique may be performed by the ECG analyzer 242 of FIG. 2 and/or the prediction module 310 of FIG. 3. The predictive modeling technique may include an LPC filter, PCA, or another predictive modeling technique. In some embodiments, the method 500 may further include preprocessing of the ECG signal prior to applying predictive modeling technique to the ECG signal. For example, the method 500 may include applying a window function (e.g., tapered cosine window) and/or a bandpass filter to the ECG signal. The preprocessing may filter out noise and other extraneous data from the digitized samples of the ECG signal.

The method 500 may further include subtracting the predicted signal from the ECG signal to provide an error signal, at 520. Subtraction of the predicted signal from the ECG signal may be performed by the ECG analyzer 242 of FIG. 2 and/or the prediction module 310 of FIG. 3. The method 500 may further include applying a bandpass filter to the error signal to generate a bandpass error signal, at 525. Application of the bandpass filter to the error signal may be performed by the ECG analyzer 242 of FIG. 2 and/or the analyzer 320 of FIG. 3.

The method 500 may further include generating decision parameters from the ECG signal, the error signal, and/or the bandpass error signal, at 530. The decision parameters may indicate characteristics of the signals that are used to classify the ECG rhythm of the patient as shockable or non-shockable. Examples of the decision parameters may include std_{S}, std_{E}, std_{EPB}, a std_{E}/std_{S} ratio, sumInvAbs_{E}, sumInvAbs_{E}, smoothInvAbs_{E}, smoothInvNormAbs_{E}, probAbsMax_{E}, probAbsNormMax_{E}, dcHilbert_{EBP}, different additional parameters, or any combination thereof. The method 500 may further include applying the decision parameters to a decision module, at 540. The decision module may include the decision module 330 of FIG. 3. The decision module may include an artificial neural network, a support vector machine, or may employ logistic regression or other techniques based on machine learning.

In some embodiments, the method 500 may further include generating probabilities associated with each of various ECG rhythms (e.g., VF, asystole, or organized electrical activity) based on the decision parameters. The method 500 may further include selecting an ECG rhythm having a highest probability.

The method 500 may further include determining whether the decision module indicates whether the ECG rhythm is shockable or non-shockable, at 550. Responsive to the decision module indicating a shockable rhythm, the method 500 may include classifying the electrocardiogram signal as a shockable rhythm, at 560. Responsive to the decision module indicating a non-shockable rhythm, the method 500 may include classifying the electrocardiogram signal as a non-shockable rhythm, at 570.

The method 400 and the method 500 may also be performed on an ECG signal clip that does not contain artifacts related to CPR or another medical procedure. The method 400 and/or the method 500 may be implemented by a field-programmable gate array (FPGA) device, an application-specific integrated circuit (ASIC), a processing unit such as a central processing unit (CPU), a digital signal processor (DSP), a controller, another hardware device, a firmware device, or any combination thereof. As an example, the method 400 and/or the method 500 may be implemented by a computing system using, for example, one or more processing units that may execute instructions for performing the method that may be encoded on a computer readable medium. The processing units may be implemented using, e.g. processors or other circuitry capable of processing (e.g. one or more controllers or other circuitry). The computer readable medium may be transitory or non-transitory and may be implemented, for example, using any suitable electronic memory, including but not limited to, system memory, flash memory, solid state drives, hard disk drives, etc. One or more processing units and computer readable mediums encoding executable instructions may be used to implement all or portions of noise filter systems, encoders, and/or encoding systems described herein.

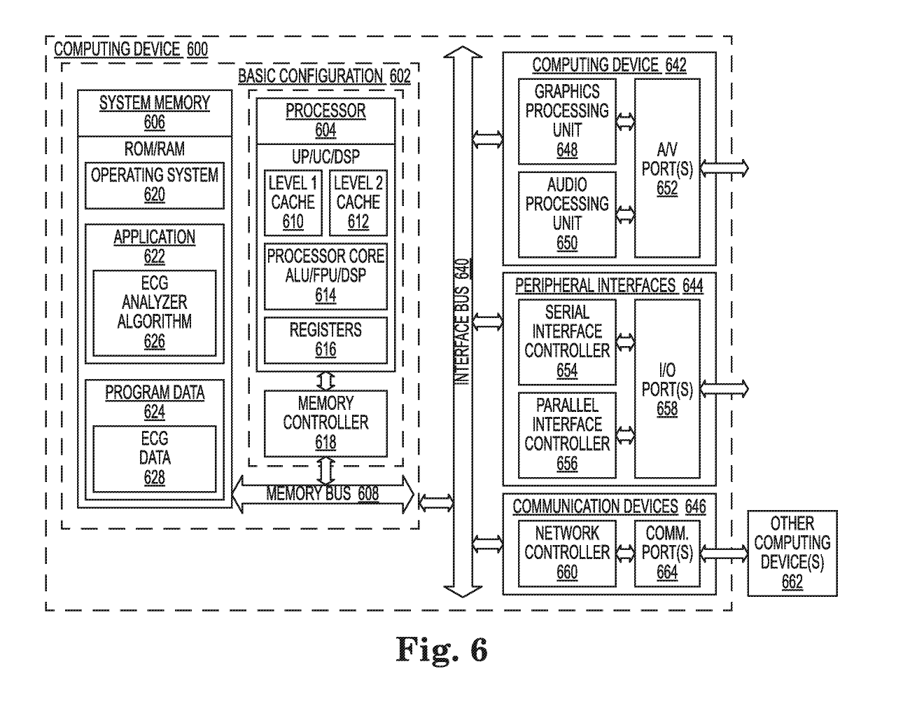

FIG. 6 is a block diagram illustrating an example computing device 600 that is arranged to implement remote display control according to at least some embodiments described herein. In a very basic configuration 602, computing device 600 typically includes one or more processors 604 and a system memory 606. A memory bus 608 may be used for communicating between processor 604 and system memory 606.

Depending on the desired configuration, processor 604 may be of any type including but not limited to a microprocessor (μP), a microcontroller (μC), a digital signal processor (DSP), or any combination thereof. Processor 604 may include one or more levels of caching, such as a level one cache 610 and a level two cache 612, a processor core 614, and registers 616. An example processor core 614 may include an arithmetic logic unit (ALU), a floating point unit (FPU), a digital signal processing core (DSP Core), or any combination thereof. An example memory controller 618 may also be used with processor 604, or in some implementations memory controller 618 may be an internal part of processor 604.

Depending on the desired configuration, system memory 606 may be of any type including but not limited to volatile memory (such as RAM), non-volatile memory (such as ROM, flash memory, etc.) or any combination thereof. System memory 606 may include an operating system 620, one or more applications 622, and program data 624. Application 622 may include an ECG analyzer 626 that is arranged to perform the functions as described herein including those described with respect to the method 400 of FIG. 4 and/or the method 500 of FIG. 5. The ECG analyzer 626 may include the AED 110 of FIG. 1, the controller 240, the ECG analyzer 242, the ECG data 262, and/or the ECG parameters 264 of FIG. 2, the ECG analyzer 300 of FIG. 3, or combinations thereof. Program data 624 may include ECG data 628 that may be useful for operation with the remote display control algorithm as is described herein. The ECG data 628 may include the ECG data 262 and/or the ECG parameters 264 of FIG. 2, and/or data from the ECG analyzer 242 of FIG. 2 and/or the ECG analyzer 300 of FIG. 3. In some embodiments, application 622 may be arranged to operate with program data 624 on operating system 620 such that implementations of convenient remote display control may be provided as described herein. This described basic configuration 602 is illustrated in FIG. 6 by those components within the inner dashed line.

Computing device 600 may have additional features or functionality, and additional interfaces to facilitate communications between basic configuration 602 and any required devices and interfaces. For example, a bus/interface controller may be used to facilitate communications between basic configuration 902 and one or more data storage devices via a storage interface bus. Data storage devices may be removable storage devices, non-removable storage devices, or a combination thereof. Examples of removable storage and non-removable storage devices include magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include volatile and nonvolatile, removable and non-removable media implemented in any method or technology for storage of information, such as computer readable instructions, data structures, program modules, or other data.

System memory 606, removable storage devices and non-removable storage devices are examples of computer storage media. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 600. Any such computer storage media may be part of computing device 600.

Computing device 600 may also include an interface bus 640 for facilitating communication from various interface devices (e.g., output devices 642, peripheral interfaces 644, and communication devices 646) to basic configuration 602 via bus/interface controller. Example output devices 642 include a graphics processing unit 648 and an audio processing unit 650, which may be configured to communicate to various external devices such as a display or speakers via one or more A/V ports 652. Example peripheral interfaces 644 include a serial interface controller 654 or a parallel interface controller 656, which may be configured to communicate with external devices such as input devices (e.g., keyboard, mouse, pen, voice input device, touch input device, etc.) or other peripheral devices (e.g., printer, scanner, etc.) via one or more I/O ports 658. An example communication device 646 includes a network controller 660, which may be arranged to facilitate communications with one or more other computing devices 662 over a network communication link via one or more communication ports 664.

The network communication link may be one example of a communication media. Communication media may typically be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR) and other wireless media. The term computer readable media as used herein may include both storage media and communication media.

Computing device 600 may be implemented as a portion of a small-form factor portable (or mobile) electronic device such as a cell phone, a personal data assistant (PDA), a personal media player device, a wireless web-watch device, a personal headset device, an application specific device, or a hybrid device that include any of the above functions. Computing device 600 may also be implemented as a personal computer including both laptop computer and non-laptop computer configurations.

It is intended that all matter contained in the above description or shown in the accompanying drawings shall be interpreted as illustrative only and not limiting. Changes in detail or structure may be made without departing from the spirit of the invention as defined in the appended claims. In addition, although various representative embodiments of this invention have been described above with a certain degree of particularity, those skilled in the art could make numerous alterations to the disclosed embodiments without departing from the spirit or scope of the inventive subject matter set forth in the specification and claims.

What is claimed is:

1. A system comprising:
a defibrillator comprising an electrocardiogram analyzer, the electrocardiogram analyzer configured to apply a prediction modeling technique to an electrocardiogram signal to generate a predicted signal, wherein the electrocardiogram signal may be captured from a patient undergoing cardiopulmonary resuscitation, wherein the electrocardiogram analyzer is further configured to subtract the predicted signal from the electrocardiogram signal to generate an error signal, wherein the electrocardiogram analyzer is further configured to classify a rhythm of the electrocardiogram signal as one of a shockable rhythm or non-shockable based on the error signal.

2. The system of claim 1, wherein the defibrillator is further configured to provide a shock voltage to a pair of electrodes responsive to a classification of the electrocardiogram signal as having a shockable rhythm.

3. The system of claim 1, wherein the electrocardiogram analyzer is further configured to generate decision parameters based on the error signal.

4. The system of claim 3, wherein the electrocardiogram analyzer is further configured to generate probabilities for a plurality of electrocardiogram rhythms associated with the electrocardiogram signal based on the decision parameters.

5. The system of claim 4, wherein the plurality of electrocardiogram rhythms includes ventricular fibrillation, asystole, and organized electrical activity.

6. The system of claim 3, wherein the decision parameters indicate at least one of energy of the error signal, energy of the error signal relative to energy of the electrocardiogram signal, frequency of common amplitudes within the error signal, indications of magnitudes of amplitudes within the error signal.

7. The system of claim 1, wherein the electrocardiogram analyzer includes a decision module that is configured to classify the rhythm of the electrocardiogram signal, wherein the decision module includes at least one of an artificial neural network, support vector machines, a logistic regression module, or another technique based on machine learning.

8. The system of claim 7, wherein the decision module is trained using a plurality of previously captured and classified electrocardiogram signals.

9. A non-transitory computer-readable medium comprising instructions that, when executed by one or more processing units, cause the one or more processing units to:
generate a residual error signal by subtracting a predicted signal from an electrocardiogram signal, wherein the electrocardiogram signal includes artifacts associated with a patient undergoing cardiopulmonary resuscitation;
generate decision parameters based on the residual error signal, wherein the decision parameters indicate characteristics of the residual error signal; and
determine a respective probability value associated with an electrocardiogram rhythm based on the decision parameters using a decision module, wherein the decision module is trained using previously captured electrocardiogram signals; and
classify the electrocardiogram signal based on the probability value.

10. The non-transitory computer-readable medium of claim 9, wherein the decision module includes an artificial neural network.

11. The non-transitory computer-readable medium of claim 9, further comprising instructions that, when executed by the one or more processing units, cause the one or more processing units to determine a respective probability value associated with each of a plurality of electrocardiogram rhythms, wherein the plurality of electrocardiogram rhythms includes ventricular fibrillation, asystole, and organized electrical activity.

12. The non-transitory computer-readable medium of claim 11, further comprising instructions that, when executed by the one or more processing units, cause the one or more processing units to select an electrocardiogram rhythm of the plurality of electrocardiogram rhythms that is associated with a highest respective probability value.

13. The non-transitory computer-readable medium of claim 9, further comprising instructions that, when executed by the one or more processing units, cause the one or more processing units to apply a predictive modeling technique to the electrocardiogram signal to generate the predicted signal.

14. The non-transitory computer-readable medium of claim 13, wherein the predictive modeling technique includes linear predictive coding.

15. A method, comprising:
applying a predictive modeling technique to an electrocardiogram signal to generate a predicted signal, wherein the electrocardiogram signal includes associated with a patient undergoing cardiopulmonary resuscitation;
subtracting the predicted signal from the electrocardiogram signal to generate an error signal; and
classifying a rhythm of the electrocardiogram signal as one of a shockable rhythm or non-shockable rhythm based on the error signal.

16. The method of claim 15, further comprising preprocessing an initial electrocardiogram signal to provide the electrocardiogram signal.

17. The method of claim 16, wherein preprocessing the initial electrocardiogram signal to provide the electrocardiogram signal comprises applying a tapered cosine window function and a bandpass filter to the initial electrocardiogram signal to provide the electrocardiogram signal.

18. The method of claim 15, further comprising applying a bandpass filter to the error signal to generate a bandpass error signal.

19. The method of claim 18, further comprising generating decision parameters associated with the error signal and the bandpass error signal, wherein the decision parameters are indicative of characteristics of the error signal and the bandpass error signal.

20. The method of claim 19, further comprising applying the decision parameters to a decision module, wherein the decision module includes at least one of an artificial neural network, vector machines, or a logistic regression module, or another technique based on machine learning.

21. The method of claim 20, wherein classifying the rhythm of the electrocardiogram signal as one of a shockable rhythm or non-shockable rhythm comprises:
generating a respective probability value associated with each of a plurality of electrocardiogram rhythms based on the decision parameters; and
selecting an electrocardiogram rhythm of the plurality of electrocardiogram rhythms that is associated with a highest respective probability value.

22. The method of claim 21, further comprising:
responsive to the selected electrocardiogram rhythm being the shockable rhythm, classifying the rhythm of the electrocardiogram as the shockable rhythm; and
responsive to the selected electrocardiogram rhythm being the non-shockable rhythm, classifying the rhythm of the electrocardiogram as the non-shockable rhythm.
